(19) United States
(12) Patent Application Publication
(10) Pub. No.: US 2016/0265029 A1
(43) Pub. Date: Sep. 15, 2016

(54) QUANTITATIVE REAL-TIME AND END-POINT COLORIMETRIC PCR DEVICE

(71) Applicant: AGENCY FOR SCIENCE, TECHNOLOGY AND RESEARCH, Connexis, Singapore (SG)

(72) Inventors: Jackie Y. YING, Singapore (SG); Saravana Kumar KUMARASAMY, Singapore (SG)

(21) Appl. No.: 15/036,398

(22) PCT Filed: Nov. 7, 2014
(86) PCT No.: PCT/SG2014/000524

§ 371 Date: May 12, 2016

(30) Foreign Application Priority Data
Nov. 12, 2013 (SG) 201308391-0

(51) Int. Cl. C12Q 1/68; G01N 21/25; G01N 21/78

(52) U.S. Cl. CPC C12Q 1/686; G01N 21/78; G01N 21/251; G01N 2201/062; G01N 2201/0638; G01N 2201/0683

(57) ABSTRACT

A colorimetric-based DNA diagnostic system which includes a detector module, a processor and a memory is provided. The detector module is disposed to record an image of a DNA sample illuminated by a light source. The memory includes computer program code which along with the memory is configured, with the processor, to perform (a) sending a signal to adjust the temperature of the DNA sample to be within an approximate temperature range over which the color of the DNA sample changes, (b) sending a signal to the detector module to capture an image of the DNA sample at defined intervals within the approximate temperature range, (c) processing the captured images to extract color information, and (d) processing the extracted color information to objectively determine a melting temperature within the approximate temperature range at which the color of the DNA sample changes.

### PRIORITY CLAIM

The present application claims priority to Singapore Patent Application No. 201308391-0, filed 12 Nov. 2013.

### FIELD OF THE INVENTION

The present invention relates to polymerase chain reaction (PCR) biological assay. In particular, it relates to a quantitative real-time colorimetric PCR system for end-point melt curve analysis.

### BACKGROUND OF THE DISCLOSURE

Genotyping has traditionally involved the use of costly assays, such as real-time PCR and DNA sequencing. Various strategies have been attempted in real-time PCR such as modifying the annealing temperature so that the PCR product is not amplified in the event of a base pair mismatch between the probe and target amplicon. Genotyping can also be performed via end-point hybridization using DNA microarray systems where wild type-specific probes and mutant-specific probes are immobilized on a solid substrate. DNA sequencing opens up the possibility for detecting mutations over a very long sequence and potentially the entire genome. However, the high cost incurred due to the use of fluorophores and fluorescence imaging devices in the aforementioned methods is a major limitation. Alternatively, the use, of fluorophores can be avoided via regular PCR, where primers can be designed such that the 3′ side falls on a mutation site so that no PCR amplification can take place if the site is indeed mutated. However, this would require manually intensive and time-consuming gel electrophoresis to be performed to verify if the PCR product has been amplified.

One conventional method demonstrated a simple and cost-effective colorimetric assay for genotyping. The assay enabled the detection of gene mutations via a melt curve analysis on single-stranded DNA (ssDNA) targets hybridized to gold nanoparticle-conjugated morpholino probes. The hybridization gave the solution a pinkish hue. However, upon melting, the ssDNA-probe solution would turn colorless. The assay was highly sensitive whereby a single base pair mutation resulted in a melting temperature difference of approximately five to twelve degrees Centigrade between the wild type and mutant. The DNA probes used were significantly less expensive than conventional fluorophore-conjugated probes. Since it was colorimetric, there was no need for an expensive and bulky light source, optical filters and high-end imaging devices. In fact, genotyping in accordance with this conventional method was as simple and straightforward as adding the DNA probe and salt to the PCR-amplified product and observing, with the naked eye, the temperature at which the pinkish hue disappears.

However, the visual assessment of color change is highly subjective, and this may result in variations to the melting temperature recorded by different operators. The interpretation may also be biased by external factors such as ambient lighting. A visual assessment also significantly limits the number of samples that can be monitored at any given time, as it may not be possible for an operator to simultaneously monitor color change in a large number of samples, unless multiple operators perform this task together. A further drawback is that the process is labor-intensive, as it requires the operator to continuously monitor color change, thus preventing him/her from performing other laboratory tasks at hand. It is also tedious and causes fatigue, which in turn adversely impacts the visual interpretation. The operator may not be able to precisely identify the melting temperature due to the subtle color change in certain cases.

A more accurate outcome could be achieved by computing the derivative of color change as in a standard fluorescence melt curve analysis, which is not possible in a visual assessment. Fluorescence-based PCR imaging technologies have thus far dominated the molecular diagnostics space, but the advent of colorimetric-based assays for real-time PCR and end-point PCR such as a melt curve analysis has underlined the need for quantitative colorimetric devices.

Thus, what is needed is a low-cost and quantitative real-time colorimetric PCR system which can perform image acquisition, image analysis and thermal cycling in a real-time PCR setting with end-point melt curve analysis. Furthermore, other desirable features and characteristics will become apparent from the subsequent detailed description and the appended claims, taken in conjunction with the accompanying drawings and this background of the disclosure.

### SUMMARY

According to the Detailed Description, colorimetric-based DNA diagnostic system is provided. The colorimetric-based DNA diagnostic system includes a detector module, a processor and a memory. The detector module is disposed to record an image of a DNA sample illuminated by a light source. The memory includes computer program code which along with the memory is configured, with the processor, to at least perform (a) sending a signal to adjust the temperature of the DNA sample to be within an approximate temperature range over which the color of the DNA sample changes, (b) sending a signal to the detector module to capture an image of the DNA sample at defined intervals within the approximate temperature range, (c) processing the captured images to extract color information, and (d) processing the extracted color information to objectively determine a melting temperature within the approximate temperature range at which the color of the DNA sample changes.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 illustrates a front right top perspective view of a colorimetric real-time and end-point polymerase chain reaction (PCR) system for point-of-care (POC) applications in accordance with present embodiments.

FIG. 2 depicts a front planar view of a bench-top PCR system in accordance with the present embodiments.

FIG. 3, comprising FIGS. 3A and 3B, illustrates the Fresnel lens and microtiter plate assembly for the PCR system of FIG. 2 in accordance with the present embodiments, wherein FIG. 3A depicts a schematic view of the Fresnel lens and microtiter plate assembly and FIG. 3B depicts a front planar view of the Fresnel lens and microtiter plate assembly.

FIG. 4 illustrates Matlab codes for initialization, imaging and termination of an imaging session of the PCR system of FIG. 2 in accordance with the present embodiments.

FIG. 5 illustrates Matlab codes for communicating with a temperature controller of the PCR system of FIG. 2 in accordance with the present embodiments.

FIG. 6 illustrates a component flow for a first temperature control scheme of the PCR system of FIG. 2 in accordance with the present embodiments.

FIG. 7 illustrates a component flow for a second temperature control scheme of the PCR system of FIG. 2 in accordance with the present embodiments.

FIG. 8, comprising FIGS. 8A and 8B, illustrates Matlab code for performing a melt curve via software control of a webcam and thermal cycler of the PCR system of FIG. 2 in accordance with the present embodiments.

FIG. 9 depicts a graph of melt curve profiles for three different single-stranded DNA probe (ssDNA-probe) hybrid solutions profiled by the PCR system of FIG. 1 in accordance with the present embodiments.

FIG. 10 depicts a graph of temperature sensing in accordance with the first and the second temperature control schemes of the PCR system of FIG. 2 in accordance with the present embodiments.

FIG. 11 depicts a graph of melt curve profiles for two identical ssDNA-probe hybrid solutions in different wells of the microtiter plate of the PCR system of FIG. 2 in accordance with the present embodiments.

FIG. 12 depicts a top planar view of a 96-well microtiter plate illuminated by LEDs in the PCR system of FIG. 2 in accordance with the present embodiments.

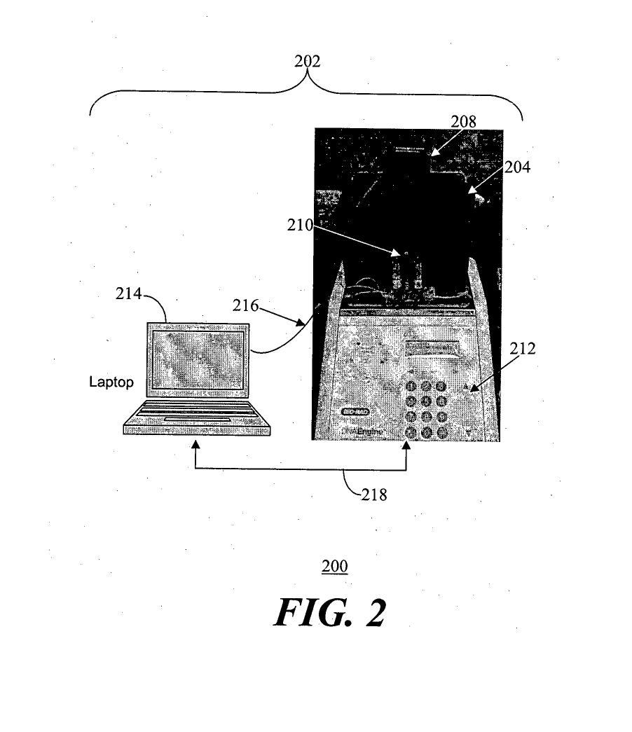

And FIG. 13 depicts a graph of color change in a melt curve analysis using red chromaticity in the PCR system of FIG. 2 in accordance with the present embodiments.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. Herein, low-cost and quantitative real-time colorimetric polymerase chain reaction (PCR) systems are presented in accordance with present embodiments. The PCR systems can perform image acquisition, image analysis and thermal cycling in both a real-time PCR setting and in end-point melt curve analysis. One embodiment is designed for point-of-care (POC) applications, whereas the other embodiment is a bench-top device for laboratory use.

Both PCR systems generally comprise (i) a color camera such as a HD Webcam C525 sold by. Logitech International S.A. of Switzerland, (ii) a Peltier heating module such as those sold by Ferrotec Corporation of California, USA, (iii) software control by a software system such as the Matlab Image Acquisition Toolbox licensed by the Mathworks, Inc. of Massachusetts, USA, (iv) a light-insulating device such as those sold by Whits Technologies of Singapore, and (v) a LED light source, such as a cool white 24 cd LED light source (e.g., C503C-WAS-CBADA151) sold by Element 14 of Singapore.

The software controls both the Peltier heating module and the color camera, such that a single-stranded DNA probe (ssDNA-probe) solution is heated from room temperature to a pre-defined temperature. At fixed temperature intervals, an image of the sample is acquired and its color information is extracted and quantified. The light-insulating device prevents ambient light from illuminating the samples, and the built-in LED is used for sample illumination so that the entire process is repeatable and not subject to fluctuations in ambient lighting.

Both PCR systems involve colorimetric genotyping assay for detection of gene mutations via a melt curve analysis on single-stranded DNA (ssDNA) targets hybridized to DNA probes. The hybrid solution, which initially has a visible color, turns colorless upon melting. However, while colorimetric assays may enable visualization of color change with the naked eye, they still entail a high degree of subjectivity where the interpretation of the instance and extent of color change vary from one individual to another.

The PCR systems in accordance with present embodiments are cost-effective since they have no moving, parts, and the various components such as the web-based camera, the Fresnel lens, the LEDs and the insulating device, are low-cost. The PCR systems are also completely automated, whereby the software provides real-time control of the Peltier heating module/thermal cycler and camera. The software also incorporates image and signal processing routines to generate the melt curve profile and precisely calculate the melting temperature. The combination of Fresnel lens, polarizing filter and LEDs ensures that the entire field of view is captured by the bench-top device, thus removing the need for a scanner. The devices also ensure that the colorimetric assays are quantitative and repeatable. Thus, the PCR systems in accordance with the present embodiments can be potentially adapted for any colorimetric assays, such as enzyme-linked immunosorbent assays (ELISA) and PCR-ELISA.

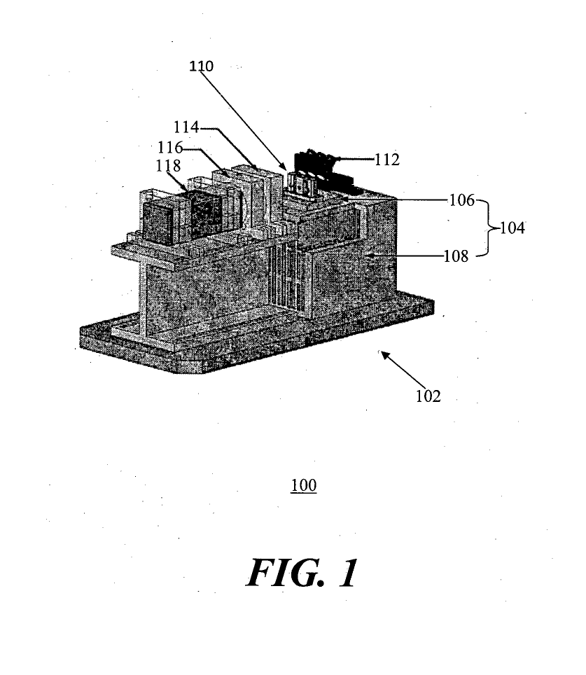

Referring to FIG. 1, a front right top perspective view 100 illustrates a colorimetric real-time and end-point PCR device 102 in accordance with the present embodiments for POC applications. The PCR device 102 includes a heating module 104 which includes a Peltier heater 106, a heat sink 108 and a copper holder 110. The PCR device 102 further includes an ultrabright white LED 112, a polarizing filter 114, a focusing lens 116 and a webcam 118. The entire setup is contained within an ambient light insulating casing (not shown). Both imaging and thermal heating/cycling are software controlled

In accordance with the present embodiments, the PCR device 102 is an integrated design for performing colorimetric genotyping at the point of care. It enables both the PCR and subsequent genotyping steps to be performed on the same platform. The battery-operated LEDs 112 provide a white broadband light source for illuminating the samples such that the resulting absorbance color (i.e., a pinkish hue) can be captured by the camera 118. As shown in the view 100, the LEDs 112 are tilted in a 45° orientation to prevent the light from saturating the camera's 118 field of view. The PCR device 102 requires a low voltage supply of five volts DC and draws a current of less than two amperes, and can potentially draw power from a car battery in a remote outdoor setting.

Several conventional fluorescence-based real-time PCR devices have been developed for POC diagnostics, however in accordance with the present embodiments a colorimetric real-time PCR device 102 is implemented. The POC PCR device 102 is better designed for portability. It can be operated by a five volt DC power supply instead of the typical twelve volt power supply. In addition, the heating module 104 has a smaller footprint for the same patient throughput of three samples in 200-μL PCR tubes, where each sample is illuminated by a dedicated one of the white LED light sources 112. Further, instead of using the typical photomultiplier tube (PMT), the focusing lens 116, excitation and emission filters 114, and a low-cost webcam 118 are employed in the PCR device 102.

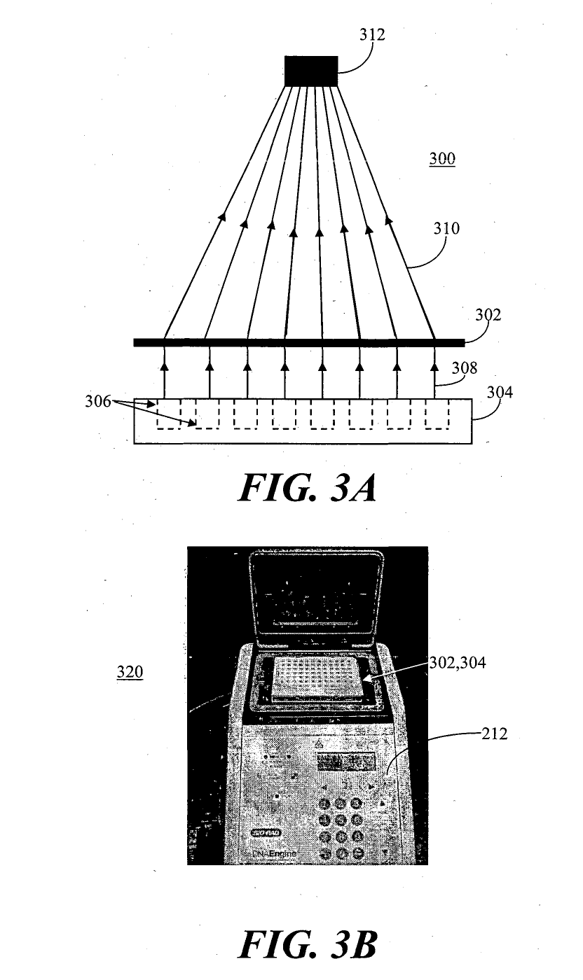

While the POC device is designed for portability, the bench-top device is designed for a larger throughput of 96 or 384 samples by interfacing the device to a conventional thermal cycler, where the heat block accommodates up to 96 or 384 samples. Referring to FIG. 2, a front planar view 200 of a colorimetric real-time and end-point bench-top PCR system 202 in accordance with the present embodiments is depicted. The PCR system 202 includes a light-insulating device 204, a built-in LED light source, a webcam 208, a Fresnel lens, a white opaque microtiter plate and 4.5 volt DC battery supply 210. A standard thermal cycler 212, such as a Bio-Rad PTC 200 thermal cycler sold by Bio-Rad Laboratories of California, USA, is used, and the imaging, the LED illumination and the thermal heating/cycling are software controlled by software on a computer 214. The webcam is powered from a USB port of the computer/laptop via a USB cable 216, whereas the LED light source can be powered either by the 4.5 volt DC battery supply 210 or alternatively by the USB port of the computer 214 via the USB cable 216. The software communicates directly with the thermal cycler 212 and the webcam 208 via a USB-to-serial port interface 218. The Fresnel lens is used together with a polarizing filter so that the entire field of view of the 96-well plate is acquired by the camera 208 without light reflections and glare from the Fresnel surface as described in more detail in accordance with FIG. 3.

Traditionally, conventional bench-top real-time PCR devices are fluorescence-based and therefore require expensive imaging components. In most cases, these devices also incorporate expensive optical scanners. In accordance with the present embodiments, a low-cost bench-top PCR system 202 including an imaging module is provided which can be potentially interfaced to various thermal cyclers commonly used in laboratories and hospitals. The PCR system 202 is designed to be cost-effective and, as such, a low-cost webcam 208 is used for imaging. The camera 208 is mounted as close as possible to the microtiter plate without compromising its coverage of the entire plate given that colorimetric signals typically have poorer contrast than fluorescence signals and that webcams 208, unlike scientific cameras, have poorer sensitivity. The camera 208, the built-in LED light source, the light insulating device 204, the battery power supply 210, the Fresnel lens and the microtiter plate function as a module (as seen in the view 200) which can be plugged into the standard thermal cycler 212 and coupled to the standard computer 214 for measuring and quantifying the colorimetric assay.

Referring to FIG. 3A, a schematic view 300 illustrates the Fresnel lens 302 and the microtiter plate 304 assembly. The Fresnel lens 302 could be a Fresnel lens such as those sold by Edmund Optics of New Jersey, USA and the white opaque microtiter plate 304 could be a white microplate such as those sold by Thermo Fisher Scientific, Inc. of Massachusetts, USA and having either 96 wells 306 or 384 wells 306. The Fresnel lens 302 enables acquisition of the entire well plate (i.e. enabling wide-angle imaging of the entire 96- or 384-well microtiter plate 304). The Fresnel lens 302 is securely fitted right above the microtiter plate 304 where light rays 308 reflected by the samples in the wells 306 are all assumed to be perpendicular to the Fresnel lens 302. The Fresnel lens 302 refracts the rays 308 such that refracted rays 310 form well-resolved points in the image-forming plane of a detector module 312 (the detector module 312 including the camera 212). Light reflections and glare from the Fresnel lens 304 is a concern in a colorimetric system which, unlike a fluorescence-based system, does not have a bandpass filter set to eliminate this problem. In accordance with the present embodiments, a polarizing filter is utilized to address this problem.

FIG. 3B depicts a front planar view 320 of the Fresnel lens 302 and the microtiter plate 304 assembly as part of the module plugged into the thermal cycler 212. In accordance with the present embodiments, the Fresnel lens 302 is located directly above the microtiter plate 304, the microtiter plate 304 having dimensions of approximately 12.8 cm×8.6 cm and the Fresnel lens 302 having a focal length of ten inches and a thickness of approximately 0.15 cm. The Fresnel lens 302 enables the base of the peripheral wells 306 in the microtiter plate 304 to be visible. In fact, a focal length of five inches should enable a better visualization of the bases of the peripheral wells 306 given a perpendicular distance of approximately 14.5 cm between the Fresnel lens 302 and the detector module 312.

The Fresnel lens 302, which is acrylic, is placed directly on the microtiter plate 304 thereby advantageously ensuring that the rays 308 emanating from each sample in the wells 306 and the LED light rays impacting each sample in the wells 306 are approximately telecentric on the object as shown in the view 300. The white opaque 96-well microtiter plate 304 provides a good contrast for the colorimetric read-out. In addition, each well 306 advantageously has a round bottom to concentrate the hybrid solution into a small area to further increase absorbance intensity. In accordance with the present embodiments, eight LED lights are positioned at the corners and sides of the ceiling within the light-insulating device 204 and the insulating device 204 is fabricated using black anodized aluminum for detection clarity. All LED lights are connected in parallel to the 4.5 volt DC battery 210 and the camera 212 is mounted in the detector module 312 at the center top portion of the module.

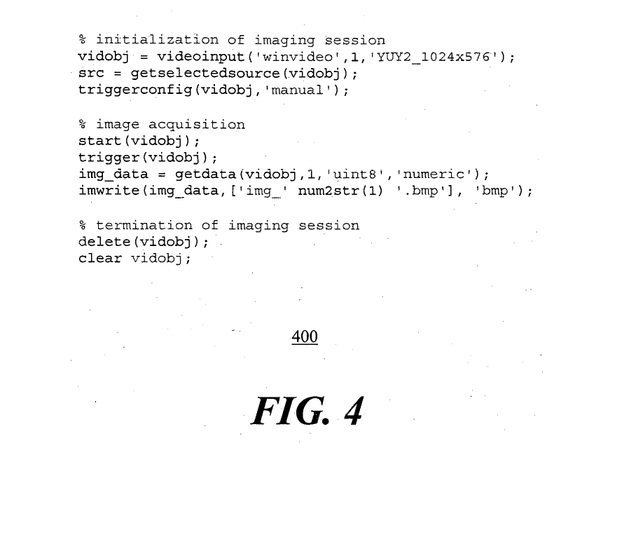

Real-time control of the camera 212 and the thermal cycler 212 and Peltier heating modules is implemented in Matlab via the USB interface 218. Referring to FIG. 4, Matlab codes 400 for initialization, imaging and termination of an imaging session of the PCR system 202 in accordance with the present embodiments is depicted. The Matlab Image Acquisition Toolbox is used to acquire both the live video feed and static images from the webcam 212 via its Windows video driver. The webcam 212 is connected to the computer/laptop 214 via the USB cable 218.

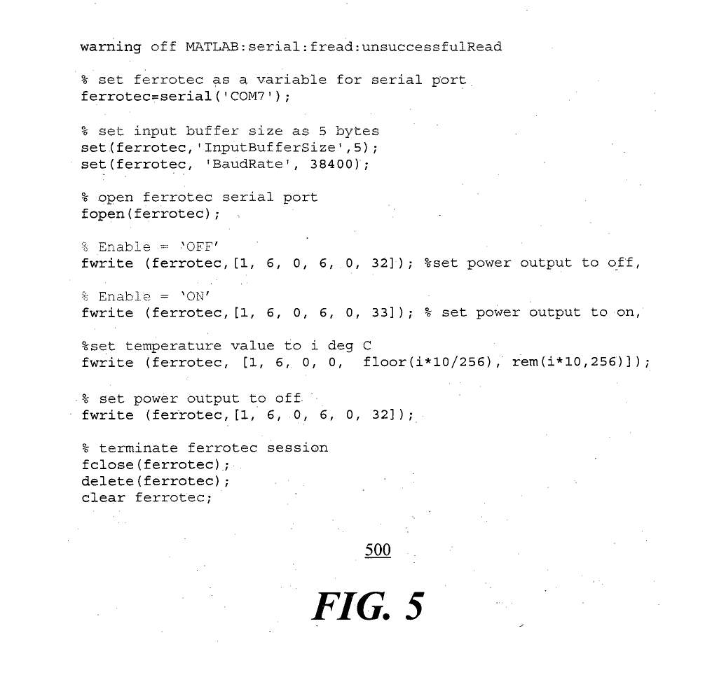

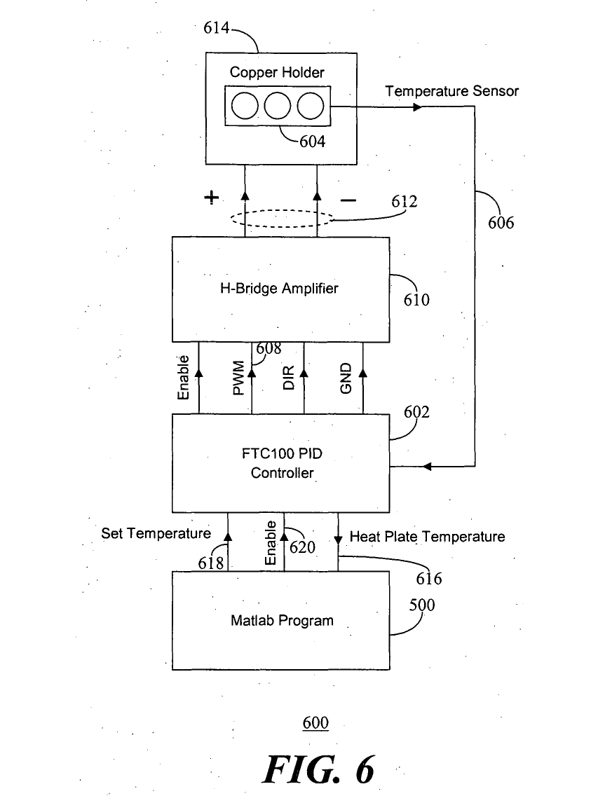

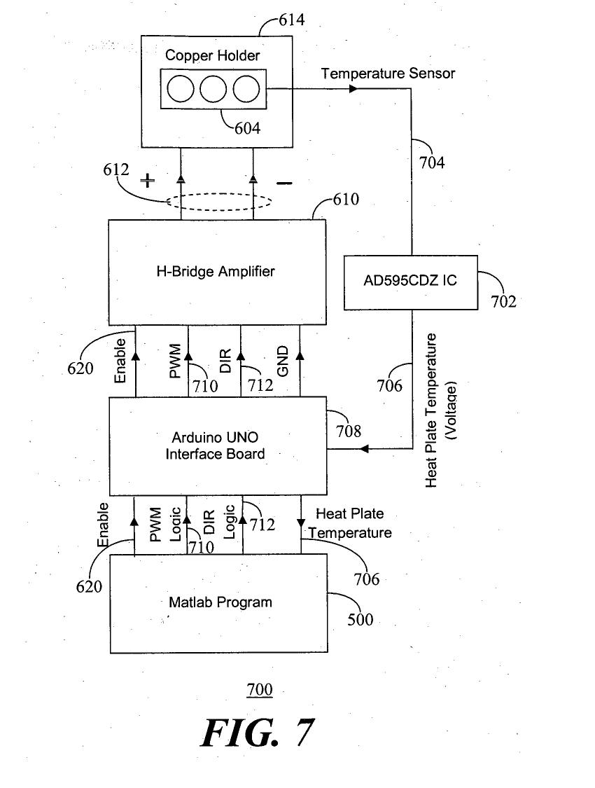

The Peltier heating module includes a temperature controller of the PCR system 202, such as a FTC 100 PID Controller sold by Accuthermo Technology Corporation of California, USA, which is also controlled via a serial port driver of the computer 214 using the serial port cable 218 together with a USB-to-serial port adapter. Referring to FIG. 5, Matlab codes 500 are depicted for communicating with the temperature controller of the PCR system 202 in accordance with the present embodiments. The codes 500 are program routines for performing real-time thermal cycling or end-point melt curve analysis. Two designs for the remote temperature control in accordance with the present embodiments are illustrated in FIGS. 6 and 7.

FIG. 6 illustrates a component flow 600 for a first temperature control scheme of the PCR 202. A temperature controller 602 senses a heat plate temperature of a heat plate 604, such as a copper holder, via a thermocouple sensor 606 and generates a pulse-width modulation (PWM) signal 608 to an amplifier 610, such as a FTA600 H-bridge amplifier board sold by Ferrotec Corporation of California, USA, which in turn generates an output voltage 612 that is fed to the Peltier heater 614. The front-end Matlab program 500 reads the heat plate temperature 616 from the temperature controller 602, and provides a set temperature signal 618 and an ENABLE signal 620 to the temperature controller 602 to initiate the heating or cooling process.

FIG. 7 illustrates a component flow 700 for a second temperature control scheme of the PCR 202. An AD595CDZ IC chip 702 sold by Analog Devices, Inc. of Massachusetts, USA senses the Peltier temperature via a K-type thermocouple sensor 704. The temperature 706 is read as an analog voltage signal, which is then read by the front-end Matlab program 500 via an input-output (I/O) Arduino UNO board 708, such as those Arduino interface boards sold by SparkFun Electronics of Colorado, USA. The Matlab program 500 implements the PID controller, and generates the PWM signal 710 and control signals (e.g., ENABLE signal 620 and DIR?? signal 712) to the H-Bridge Amplifier board 610, again via the interface board 708, for regulating the output voltage 612 to the Peltier heater 614.

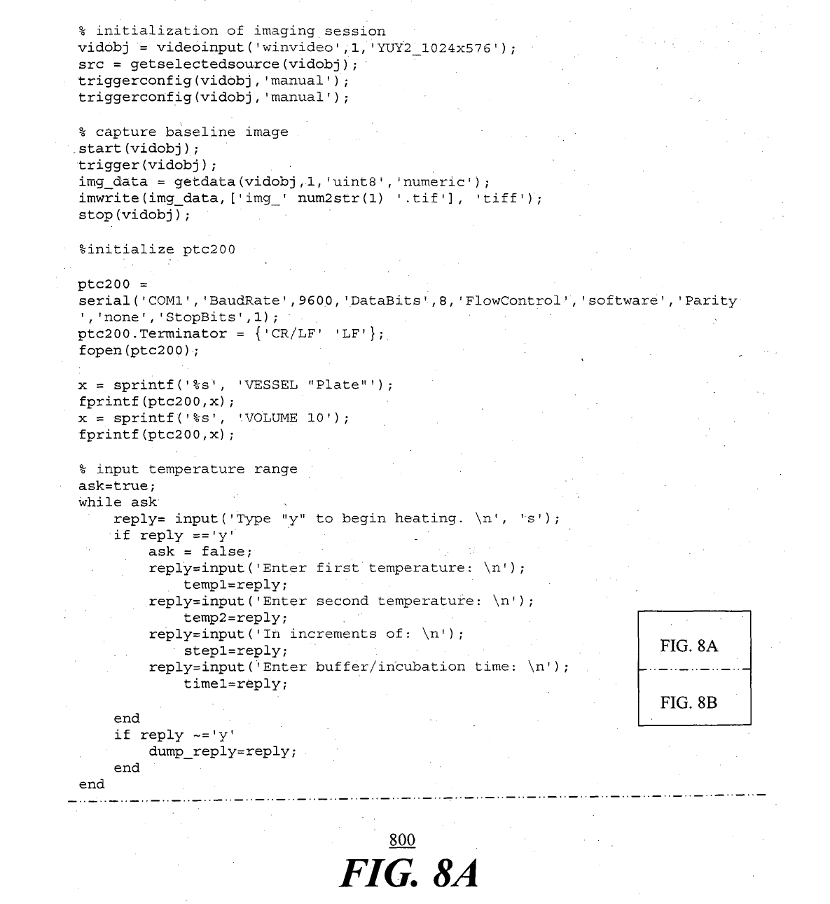

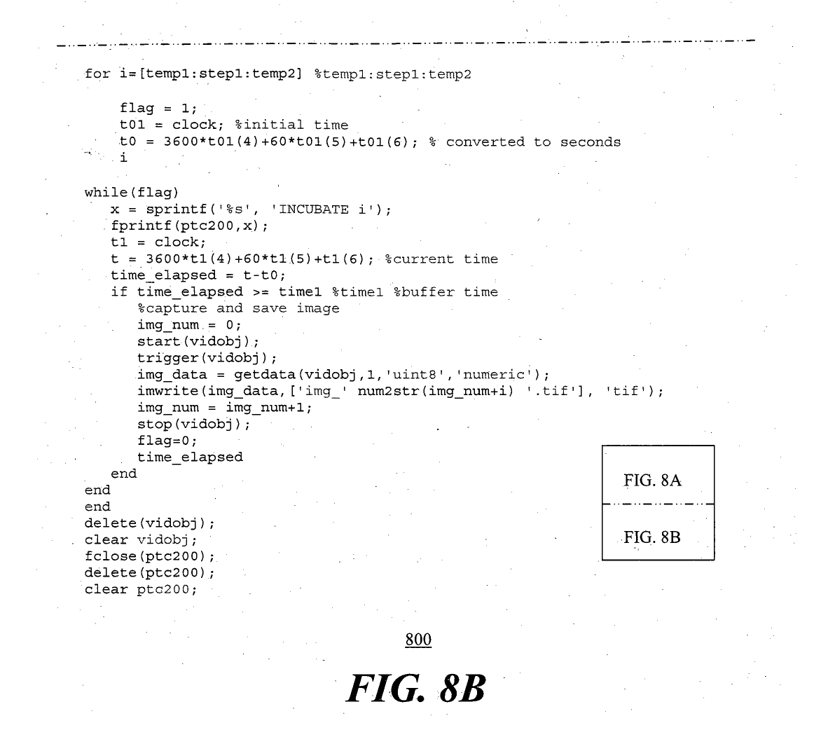

FIG. 8, comprising FIGS. 8A and 8B, illustrates Matlab code 800 for performing a melt curve via software control of the webcam 208 and the thermal cycler 212 of the PCR system 202 in accordance with the present embodiments. A user is initially prompted to input a temperature range and increment, as well as a duration for which the samples are held at a given temperature for the melt curve analysis. The front-end Matlab program 800 remotely controls the thermal cycler 212, by instructing the thermal cycler 212 to cycle through the inputted temperatures where the holding time for each temperature is as specified by the user. When the holding time is over, an image of the entire microtiter plate 304 is captured by the webcam 208 and stored in the computer 214 hard disk. This cycle is repeated for each temperature.

A melt curve is a x-y plot whereby the y-axis represents the relative absorbance unit (a.u.) and the x-axis represents temperature in ° C. A melting temperature (Tₘ) is defined as the temperature at which the ssDNA-probe hybrid solution changes from a pinkish hue to colorless.

The color information is extracted by first converting the acquired images into the luminance (Y)—blue chrominance (C_{b})—red chrominance (Cᵣ) or YC_{b}Cᵣ color space using the Matlab Image Processing Toolbox. This is done to decouple color from the luminance information. Subsequently, the red chrominance information is extracted as a proxy to monitor the change in color since red is a dominant component in the pinkish hue of the hybrid solution. Given that the melt curve is represented by the red chrominance Cᵣ vs. temperature T, the melting temperature Tₘ is defined as the point on the melt curve at which

$$- \frac{d {C}_{r}}{d T}$$

is a maxima.

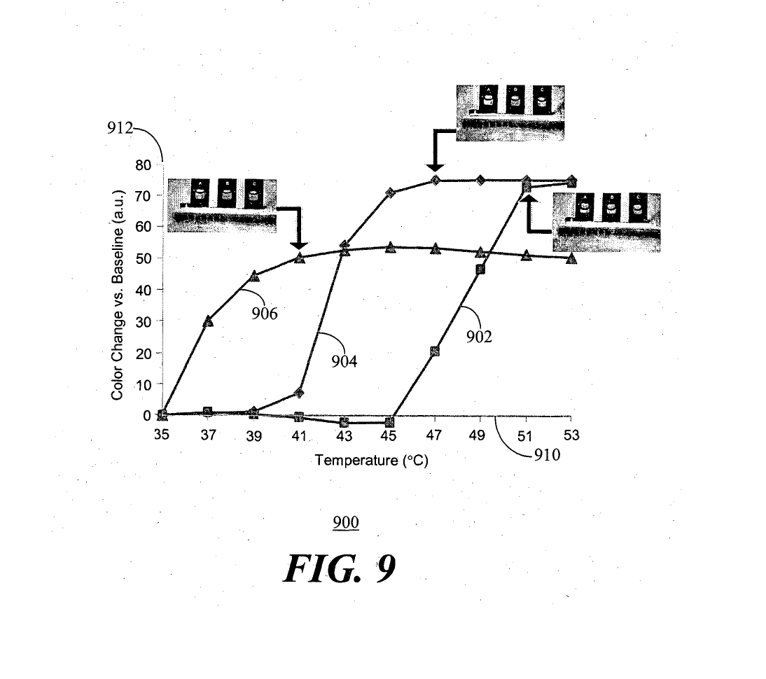

FIG. 9 depicts a graph 900 of melt curve profiles 902, 904, 906 for three different ssDNA-probe hybrid solutions profiled by the PCR system 102. The temperature is plotted along the x-axis 910 and the decrease in the value of the red-channel vs. a baseline (i.e. value at 35° C.) is plotted along the y-axis 912 in relative absorbance units. As seen from the graph 900, the melting temperature (Tₘ) ranges from 35-53° C. at an increment of 2° C. It can also be observed that the color change is gradual and occurs over 6-8° C. The POC PCR system 102 enables a high contrast imaging of the three samples where the contrast between the pinkish hue and the background or colorless solution is visually distinct.

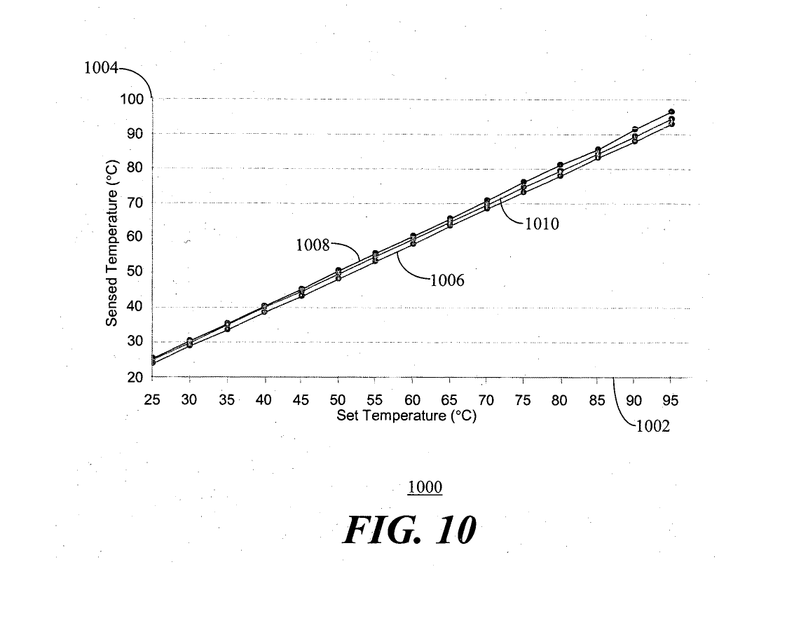

FIG. 10 depicts a graph 1000 of temperature sensing in accordance with the first and the second temperature control schemes of the PCR system 202, where a set temperature is plotted along the x-axis 1002 and a sensed temperature is plotted along the y-axis 1004. The thermal sensing unit 606 used in the first thermal sensing scheme is plotted along a trace 1006, the thermal sensing unit 704 used in the second thermal sensing scheme is plotted along a trace 1008, a commercial K-type reference thermocouple, such as a TM-947SD sold by Lutron Electronic enterprise Company, Ltd. of Taipei, Taiwan is plotted along a trace 1010. It can be seen that the thermal sensing units 606, 704 have linear profiles 1006, 1008 across the entire temperature range of 25-95° C. and their readings correlate closely with the commercial reference thermocouple (the trace 1010) with a maximum error margin of about ±2° C. at 95° C. The set temperature along the x-axis is defined by a commercial thermomixer such as those sold by Comfort Series of Eppendorf, Germany).

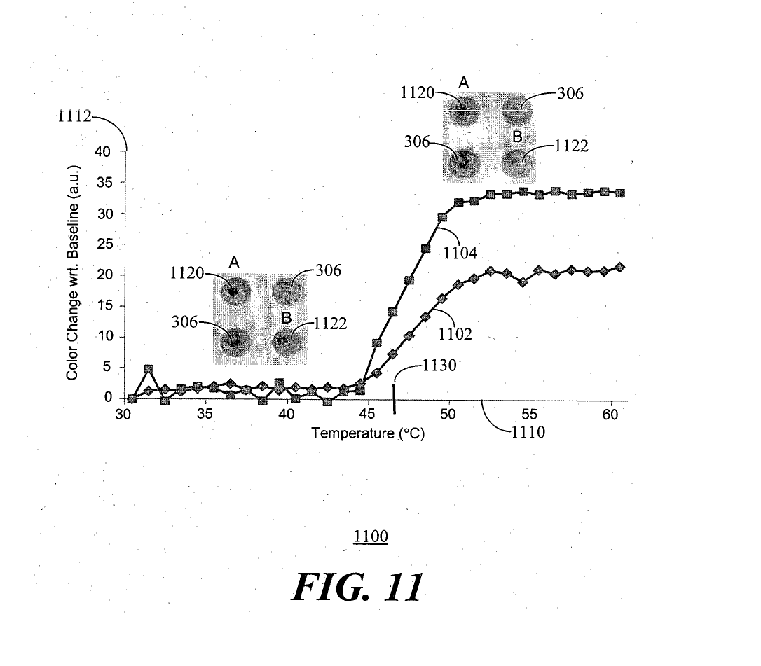

FIG. 11 depicts a graph 1100 of melt curve profiles 1102, 1104 for two identical ssDNA-probe hybrid solutions in different wells 306 of the microtiter plate 304 of the PCR system 202. The temperature is plotted along the x-axis 1110 and the decrease in the value of the red-channel vs. a baseline (i.e. value at 35° C.) is plotted along the y-axis 1112 in relative absorbance units. Ambient lighting was allowed inside the light insulating device 204 by raising the light insulating device 204 by approximately ten centimeters from the thermal cycler 212 heat plate 614. Two (Well A 1120 and Well B 1122) out of the four wells 306 were loaded with identical ssDNA-probe hybrid solutions. Although the magnitude of color change in the two wells 1120, 1122 is different, the melting temperature 1130 (˜47° C.) is the same.

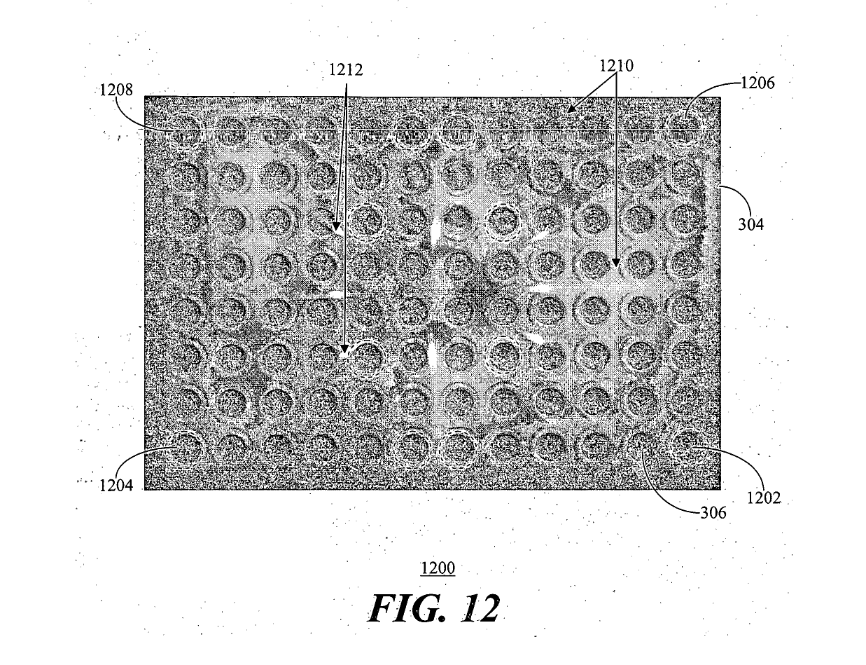

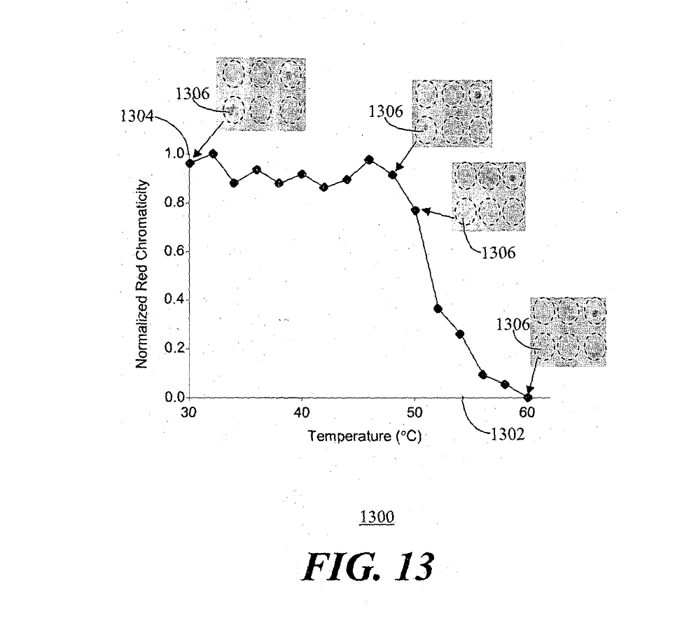

FIG. 12 depicts a top planar view 1200 of the 96-well microtiter plate 304 illuminated by LEDs fitted in the light-insulating device 204 in the PCR system 202. An improvement in signal contrast is observed with respect to the wells 1120, 1122 (FIG. 1) that were exposed to ambient light. However, the contrast is noticeably weaker in the four corner wells 1202, 1204, 1206, 1208. This is attributed to the partial occlusion of the sample in these wells (see for example, the corner well 1208). Non-uniform illumination 1210 and light reflection hot spots 1212 are also observed in the view 1200. The non-uniform illumination 1210 can be addressed by adopting a ring-like configuration of the LEDs around the optical axis of the camera 208. Alternatively, increasing the distance of the LEDs from the microtiter plate 304 and/or increasing the number of LEDs fitted promotes better light uniformity, as this increases the overlap of individual LED light projections onto the microtiter plate 304. The reflection hot spots 1212 are attributed to light bouncing off the Fresnel lens 302 and this can be addressed by incorporating a polarizing filter along the optical axis. The partial occlusion of the samples in the peripheral wells 1202, 1204, 1206, 1208 can be addressed by using a Fresnel lens 302 with stronger refractive power, i.e. a shorter focal length.

Referring to FIG. 13, a graph 1300 of automated color change in a melt curve analysis using red chromaticity in the PCR system 202 is depicted. Red chromaticity is similar to the red channel information extracted in the graph 1100 (FIG. 11), except that it is robust against variations in luminance. The red-green-blue (RGB) color space of the original image is first transformed to the luminance-blue chrominance-red chrominance (YC_{b}Cᵣ) color space, after which red chromaticity (Cᵣ) is extracted and normalized against the baseline, which in this case corresponds to the value at 30° C. The temperature is plotted along the x-axis 1302 and the normalized red chromaticity is plotted along the y-axis 1304. As the color changes from pinkish to colorless, the red chromaticity in a well 1306 decreases, evidencing that the use of red chromaticity (Cᵣ) advantageously increases contrast while reducing the influence of non-uniform illumination on the colorimetric signal.

Thus, it can be seen that systems for low-cost, rapid, automated and colorimetric-based genotyping devices have been provided for both POC and bench-top use. Although the POC device has limited throughput, whereby three DNA samples can be analyzed at one go, it is portable and can be operated by a battery. In contrast, the bench-top device has a high throughput as it leverages on a standard thermal cycler format, but it is meant for laboratory use.

Further, the present embodiments enable the generation of melt curves and localization of melting temperature. The arrangement of the LEDs is crucial in ensuring uniform illumination of the field of view, i.e. the 96-well microtiter plate 304. Instead of placing the LEDs at the corners and sides of the ceiling within the light-insulating device 204, a ring-like configuration of the LEDs around the optical axis of the camera 208 results in more uniform illumination. In addition, increasing the distance of the LEDs from the microtiter plate 304 and/or increasing the number of LEDs fitted would increase the overlap of individual LED light projections onto the microtiter plate 304, and this in turn would promote better light uniformity.

Also, in accordance with the present embodiments, the Fresnel lens 302 provides a cost-effective method for imaging the entire microtiter plate without the need for a scanning system. Internal reflections and glare from the glossy surface of the Fresnel lens 302, which may adversely affect the colorimetric read-out, can be removed or significantly reduced by incorporating a polarizing filter at the inlet to the camera 208. The transmission loss due to the polarizing filter can be offset by increasing the number of LEDs. Alternatively, the LEDs can be positioned in a manner such that the reflections do not occlude the wells.

While Fresnel lens 302 with a focal length of ten inches may only enable partial visualization of the well bases at the corners of the microtiter plate, using a Fresnel lens of a shorter focal length or increasing the distance from the microtiter plate 304 to the camera 208 advantageously brings the bases of peripheral wells 1202, 1204, 1206, 1208 within the field of view of the camera 208. While exemplary embodiments have been presented in the foregoing detailed description of the invention, it should be appreciated that a vast number of variations exist.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

1. A colorimetric-based DNA diagnostic system comprising:
a detector module disposed to record an image of a DNA sample illuminated by a light source;
a processor; and
a memory including computer program code, wherein the memory and the computer program code are configured, with the processor, to at least perform:
sending a signal to adjust the temperature of the DNA sample to be within an approximate temperature range over which the color of the DNA sample changes;
sending a signal to the detector module to capture an image of the DNA sample at defined intervals within the approximate temperature range;
processing the captured images to extract color information; and
processing the extracted color information to objectively determine a melting temperature, within the approximate temperature range, at which the color of the DNA sample changes.

2. The colorimetric-based DNA diagnostic system in accordance with claim 1, wherein the memory and the computer program code are configured, with the processor, to further perform obtaining the melting temperature using a differentiation mathematical operation on the extracted color information.

3. The colorimetric-based DNA diagnostic system in accordance with claim 2, wherein the memory and the computer program code are configured, with the processor, to further perform
constructing a melt curve using the extracted color information; and
analysing the melt curve with the differentiation mathematical operation to obtain the melting temperature.

4. (canceled)

5. The colorimetric-based DNA diagnostic system in accordance with claim 1, wherein the memory and the computer program code are configured, with the processor, to further perform objective determination of the melting temperature from a portion of the extracted color information belonging to a wavelength range that the DNA sample color falls within.

6. (canceled)

7. The colorimetric-based DNA diagnostic system in accordance with claim 1 where the image is captured at an annealing or elongation operation at every polymerase chain reaction (PCR) cycle that the DNA sample undergoes.

8. The colorimetric-based DNA diagnostic system in accordance with claim 1, wherein the melting temperature is objectively determined from images of the DNA sample captured while the temperature of the DNA sample is adjusted over the approximate temperature range.

9. The colorimetric-based DNA diagnostic system in accordance with claim 1, wherein the melting temperature is objectively determined after all images of the DNA sample, over the approximate temperature range, are captured.

10. The colorimetric-based DNA diagnostic system in accordance with claim 1, further comprising a heating module for the DNA sample, wherein the heating module receives the signal to adjust the temperature of the DNA sample to be within the approximate temperature range over which the color of the DNA sample changes.

11. The colorimetric-based DNA diagnostic system in accordance with claim 10, further comprising a temperature control device with which the processor communicates to control the temperature of the DNA sample, the temperature control device coupled to the heating module and the processor.

12. The colorimetric-based DNA diagnostic system in accordance with claim 1, further comprising
a sensor to detect the temperature of the DNA sample, wherein the memory and the computer program code are configured, with the processor, to further perform
sending the signal to adjust the temperature of the DNA sample to be within the temperature range over which the color of the DNA sample changes, when the temperature of the DNA sample read from the sensor is not within the temperature range.

13. The colorimetric-based DNA diagnostic system in accordance with claim 1, further comprising a light source disposed to illuminate the DNA sample.

14. The colorimetric-based DNA diagnostic system in accordance with claim 13, further comprising a lens located upstream of the DNA sample and before the detector module lens.

15. The colorimetric-based DNA diagnostic system in accordance with claim 14, further comprising a polarizing filter provided at the detector module lens.

16. The colorimetric-based DNA diagnostic system in accordance with claim 15, further comprising a polarizing filter provided at the light source, wherein the polarizing filter provided at the detector module lens and the polarizing filter provided at the light source are orientated to establish a Brewster angle that attenuates reflection occurring at the lens located upstream of the DNA sample and before the detector module.

17. The colorimetric-based DNA diagnostic system in accordance with claim 16, further comprising a light insulator to cover the light source, the lens, the polarizing filter provided at the detector module lens, at least the detector module lens and the heating module.

18. The colorimetric-based DNA diagnostic system in accordance with claim 17, wherein the light insulator further covers the polarizing filter provided at the light source.

19. The colorimetric-based DNA diagnostic system in accordance with claim 15, further comprising a light insulating enclosure within which the lens, the polarizing filter provided at the detector module lens, at least the detector module lens and the heating module are disposed.

20. The colorimetric-based DNA diagnostic system in accordance with claim 19, wherein the light source is located external to the light insulating enclosure and tilted relative to an axis along which the DNA sample is orientated.

21-24. (canceled)

25. The colorimetric-based DNA diagnostic system in accordance with claim 12, wherein the light source is one or more white LEDs.

26. (canceled)

27. (canceled)

28. The colorimetric-based DNA diagnostic system in accordance with claim 14, wherein the lens is a Fresnel lens.

29-33. (canceled)
